(19) United States
(12) Patent Application Publication
(10) Pub. No.: US 2014/0066770 A1
(43) Pub. Date: Mar. 6, 2014

(54) ULTRASOUND DIAGNOSTIC APPARATUS AND IMAGE ACQUISITION METHOD USING ULTRASONIC WAVES

(71) Applicants: Yoshinobu Watanabe, Kanagawa (JP); Katsumi Satake, Kanagawa (JP); Kinya Hasegawa, Kanagawa (JP)

(72) Inventors: Yoshinobu Watanabe, Kanagawa (JP); Katsumi Satake, Kanagawa (JP); Kinya Hasegawa, Kanagawa (JP)

(73) Assignee: PANASONIC CORPORATION, Osaka (JP)

(21) Appl. No.: 14/115,407

(22) PCT Filed: May 25, 2012
(86) PCT No.: PCT/JP2012/003426

§ 371 Date: Nov. 4, 2013

(30) Foreign Application Priority Data
May 30, 2011 (JP) 2011-119978

(51) Int. Cl. A61B 8/08; A61B 8/00; A61B 8/14

(52) U.S. Cl. CPC A61B 8/5246; A61B 8/14; A61B 8/4254; A61B 8/5207; A61B 8/466; A61B 8/0891
USPC 600/443

(57) ABSTRACT

This ultrasonic diagnostic apparatus includes: a probe position information obtaining section which obtains information about the position of a probe that has gotten a received signal from each point; a feature extracting and computing section which generates, based on the received signal with which the positional information has been associated, information to compose a three-dimensional image including a object of measurement; a feature data comparing section which stores information to compose a three-dimensional image that was gotten in the past and information about the position of the object of measurement, of which the characteristic was measured in the past, compares the information to compose the three-dimensional image that was gotten in the past to information to compose a three-dimensional image being gotten currently, and gets the positional information of the object of measurement, of which the characteristic was measured in the past, incorporated into the information to compose the three-dimensional image being gotten currently; and an image display processing section which performs processing to display a three-dimensional image based on the information to compose the three-dimensional image being gotten currently, the positional information of the object of measurement, of which the characteristic was measured in the past and which has been incorporated by the feature data comparing section, and information about the current position of the probe.

### TECHNICAL FIELD

The present invention relates to an ultrasonic diagnostic apparatus and a method of getting an image using an ultrasonic wave.

### BACKGROUND ART

An ultrasonic diagnostic apparatus is used to collect information to be used for medical purposes, e.g., to display the state of a blood vessel or a viscera in a human body. Such an ultrasonic diagnostic apparatus includes a probe, a transmitting section, a receiving section, a tomographic image generating section and a display section as its major components, and gets a tomographic image, which has been generated by the tomographic image generating section, displayed on the display section.

However, just by getting such a tomographic image, representing a blood vessel or the viscera in a human body, displayed by an ultrasonic diagnostic apparatus, sometimes it may not be a sufficient amount of information to be used effectively for medical purposes. That is the case particularly if it is necessary to carry out a medical checkup using an ultrasonic diagnostic apparatus on a regular basis by getting a tomographic image from the same region each time or if a tomographic image needs to be gotten from a predetermined region in a blood vessel or the viscera non-regularly.

For example, in diagnosing arterial sclerosis, a tomographic image representing a carotid artery is gotten by an ultrasonic diagnostic apparatus and the intima-media thickness (which will be abbreviated herein as “IMT”) of the carotid artery is measured based on the tomographic image. In order to know the degree of advancement of the arterial sclerosis or the status of treatment by measuring the IMT, it is recommended that the diagnosis or inspection be made based on a tomographic image gotten each time from the same region in the carotid artery.

For that reason, even though it is not dedicated to measuring the IMT, some people proposed a technique for increasing the value of such information to be used for medical purposes by combining a tomographic image gotten by an ultrasonic diagnostic apparatus with a three-dimensional image gotten by an X-ray CT scanner (see Patent Document No. 1, for example).

### CITATION LIST

### Patent Literature

PCT International Application Publication No. 2006/59668

### SUMMARY OF INVENTION

### Technical Problem

In the example of Patent Document No. 1 mentioned above, the tomographic image gotten by the ultrasonic diagnostic apparatus and the three-dimensional image gotten by the X-ray CT scanner need to be combined with each other. For that purpose, three-dimensional image data which has been composed by retrieving a saved image that has been gotten by the X-ray CT scanner and stored and saved in advance and the tomographic image which has been gotten by transmitting and receiving an ultrasonic wave to/from a certain region of the subject should be matched to each other. However, it would be very difficult to get that matching work done if the positions, tilt angles and moving directions of the probe were different when those tomographic images are gotten.

Speaking more specifically, the positional information of the tomographic image gotten by the ultrasonic diagnostic apparatus and the positional information of the three-dimensional image gotten by the X-ray CT scanner have been generated by these two different apparatuses. That is why to match those two pieces of positional information to each other, a reference point should be specified for each of those images and their two reference points should be matched to each other. However, this image matching work should be a troublesome one if their reference points set or image cropping areas were different from each other or if the cropped images had different sizes.

Particularly if the tomographic image were gotten by the ultrasonic diagnostic apparatus in a tilted position, it would be extremely difficult to match the tomographic image gotten in such a tilted position to the three-dimensional image data gotten by the X-ray CT scanner. That is why the conventional ultrasonic diagnostic apparatus has been far from coming in handy.

It is therefore an object of the present invention to provide an ultrasonic diagnostic apparatus which will come in much handier so as to allow the user to set the probe at a predetermined position just by performing a series of simple operations and get those images matched easily and also provide a method of getting an image using ultrasonic waves.

### Solution to Problem

An ultrasonic diagnostic apparatus according to an aspect of the present invention is connectable to a probe that transmits and receives an ultrasonic wave and that converts its echo signal into an electrical signal and measures a characteristic of a subject in a predetermined region of its object of measurement. The apparatus includes: a probe driving section which performs transmission processing to transmit the ultrasonic wave and reception processing to generate a received signal based on the electrical signal by driving the probe and which gets a received signal from each point of the subject including the object of measurement; a probe position information obtaining section which obtains positional information about the probe that has gotten the received signal from each point; an image generation control section which associates the received signal from each point with the positional information; a feature extracting and computing section which generates, based on the received signal with which the positional information has been associated, information to compose a three-dimensional image covering the object of measurement; a feature data comparing section which stores information to compose a three-dimensional image that was gotten in the past and information about the position of the object of measurement, of which the characteristic was measured in the past, compares the information to compose the three-dimensional image that was gotten in the past to information to compose a three-dimensional image being gotten currently, and gets the positional information of the object of measurement, of which the characteristic was measured in the past, incorporated into the information to compose the three-dimensional image being gotten currently; an image display processing section which performs processing to display a three-dimensional image based on the information to compose the three-dimensional image being gotten currently, the positional information of the object of measurement, of which the characteristic was measured in the past and which has been incorporated by the feature data comparing section, and information about the current position of the probe; and a display section which displays the output of the image display processing section.

Another ultrasonic diagnostic apparatus according to an aspect of the present invention is connectable to a probe that transmits and receives an ultrasonic wave and that converts its echo signal into an electrical signal and measures a characteristic of a subject in a predetermined region of its object of measurement. The apparatus includes: a probe driving section which performs transmission processing to transmit the ultrasonic wave and reception processing to generate a received signal based on the electrical signal by driving the probe and which gets a received signal from each point of the subject including the object of measurement; a probe position information obtaining section which obtains positional information about the probe that has gotten the received signal from each point; an image generation control section which generates tomographic image information based on the received signal from each point and which associates the tomographic image information with the positional information; a feature extracting and computing section which generates, based on the tomographic image information with which the positional information has been associated, information to compose a three-dimensional image covering the object of measurement; a feature data comparing section which stores information to compose a three-dimensional image that was gotten in the past and information about the position of the object of measurement, of which the characteristic was measured in the past, compares the information to compose the three-dimensional image that was gotten in the past to information to compose a three-dimensional image being gotten currently, and gets the positional information of the object of measurement, of which the characteristic was measured in the past, incorporated into the information to compose the three-dimensional image being gotten currently; an image display processing section which performs processing to display a three-dimensional image based on the information to compose the three-dimensional image being gotten currently, the positional information of the object of measurement, of which the characteristic was measured in the past and which has been incorporated by the feature data comparing section, and information about the current position of the probe; and a display section which displays the output of the image display processing section.

The image display processing section superimposes the information about the current position that has been obtained by the probe position information obtaining section on the three-dimensional image based on the information to compose the three-dimensional image being gotten currently and then displays the three-dimensional image with the current position information on the display section.

The ultrasonic diagnostic apparatus includes a storage section which stores the information to compose the three-dimensional image that was gotten in the past and the positional information of the object of measurement, of which the characteristic was measured in the past.

If the feature data comparing section has found that the probe's current position with respect to the object of measurement matches the position of the object of measurement, of which the characteristic was measured in the past, the feature extracting and computing section instructs the image display processing section to give a sign of their match on the display section.

The feature extracting and computing section extracts information to compose the three-dimensional image of the object of measurement from pieces of information to compose the three-dimensional image by reference to information about the structure of the object of measurement. The image display processing section gets the information to compose the three-dimensional image of the object of measurement, which has been extracted by the feature extracting and computing section, displayed on the display section.

The feature data comparing section compares the information to compose the three-dimensional image that was gotten in the past and the information to compose the three-dimensional image being gotten currently by reference to the information about the structure of the object of measurement.

The object of measurement is a carotid artery and the characteristic to measure is a characteristic of a blood vessel.

The characteristic of the blood vessel to measure is its IMT.

The feature data comparing section compares the information to compose the three-dimensional image that was gotten in the past to the information to compose the three-dimensional image being gotten currently based on the structure of a common carotid artery branching portion where the common carotid artery of the carotid artery branches into an inner carotid artery and an outer carotid artery.

The feature data comparing section compares the information to compose the three-dimensional image that was gotten in the past to the information to compose the three-dimensional image being gotten currently based on the blood vessel diameter of the carotid artery.

An image getting method according to an aspect of the present invention is a method for getting an image using an ultrasonic wave which is connectable to a probe that transmits and receives the ultrasonic wave and that converts its echo signal into an electrical signal and which measures a characteristic of a subject in a predetermined region of its object of measurement. The method includes the steps of: (A) performing transmission processing to transmit the ultrasonic wave and reception processing to generate a received signal based on the electrical signal by driving the probe and getting a received signal from each point of the subject including the object of measurement; (B) obtaining positional information about the probe that has gotten the received signal from each point; (C) associating the received signal from each point, including the object of measurement, with the positional information; (D) generating, based on the received signal with which the positional information has been associated, information to compose a three-dimensional image covering the object of measurement; (E) comparing the information to compose the three-dimensional image that was gotten in the past to information to compose a three-dimensional image being gotten currently, and getting the positional information of the object of measurement, of which the characteristic was measured in the past, incorporated into the information to compose the three-dimensional image being gotten currently; and (F) performing processing to display a three-dimensional image based on the information to compose the three-dimensional image being gotten currently, the positional information of the object of measurement, of which the characteristic was measured in the past and which has been incorporated by the feature data comparing section, and information about the current position of the probe.

Another image getting method according to an aspect of the present invention is a method for controlling an ultrasonic diagnostic apparatus which is connectable to a probe that transmits and receives an ultrasonic wave and that converts its echo signal into an electrical signal and which measures a characteristic of a subject in a predetermined region of its object of measurement. The method includes the steps of: (A′) performing transmission processing to transmit the ultrasonic wave and reception processing to generate a received signal based on the electrical signal by driving the probe and getting a received signal from each point of the subject including the object of measurement; (B′) obtaining positional information about the probe that has gotten the received signal from each point; (C′) generating tomographic image information based on the received signal from each point, including the object of measurement, and associating the tomographic image information with the positional information; (D′) generating, based on the tomographic image information with which the positional information has been associated, information to compose a three-dimensional image covering the object of measurement; (E′) comparing the information to compose the three-dimensional image that was gotten in the past to information to compose a three-dimensional image being gotten currently, and getting the positional information of the object of measurement, of which the characteristic was measured in the past, incorporated into the information to compose the three-dimensional image being gotten currently; and (F′) performing processing to display a three-dimensional image based on the information to compose the three-dimensional image being gotten currently, the positional information of the object of measurement, of which the characteristic was measured in the past and which has been incorporated by the feature data comparing section, and information about the current position of the probe.

### Advantageous Effects of Invention

An ultrasonic diagnostic apparatus and method of getting an image using an ultrasonic wave according to the present disclosure can provide an ultrasonic diagnostic apparatus which will come in much handier so as to allow the user to set the probe at a predetermined position just by performing a series of simple operations.

### BRIEF DESCRIPTION OF DRAWINGS

FIG. 1 is a block diagram generally illustrating an embodiment of an ultrasonic diagnostic apparatus according to the present invention.

FIG. 2(a) is a perspective view illustrating the appearance of the ultrasonic diagnostic apparatus shown in FIG. 1 and FIG. 2(b) is a perspective view illustrating the main portion of its positional information obtaining section.

FIG. 3 is a detailed block diagram of the ultrasonic diagnostic apparatus shown in FIG. 1.

FIG. 4 is a flowchart showing the procedure of measuring the IMT for the first time using the ultrasonic diagnostic apparatus shown in FIG. 1.

FIG. 5(a) illustrates how a probe is brought into contact with a subject and FIG. 5(b) shows a tomographic image of a region including a carotid artery.

FIG. 6(a) illustrates how to make hand scanning on the surface of the nape, and FIG. 6(b) illustrates examples of three-dimensional images of the carotid artery to be displayed on a display section.

FIG. 7 illustrates a three-dimensional image showing a region where the IMT of the carotid artery has been measured.

FIG. 8 is a flowchart showing the procedure of measuring the IMT for the second time and on using the ultrasonic diagnostic apparatus shown in FIG. 1.

FIGS. 9(a) to 9(d) illustrate how to match a three-dimensional image being gotten currently and a three-dimensional image that was gotten in the past with each other while the IMT is being measured.

FIGS. 10(a) to 10(d) illustrate three-dimensional images to be displayed on the display section when the IMT is measured for the second time and on.

### DESCRIPTION OF EMBODIMENTS

Hereinafter, embodiments of an ultrasonic diagnostic apparatus and method of getting an image using an ultrasonic wave according to the present invention will be described with reference to the accompanying drawings.

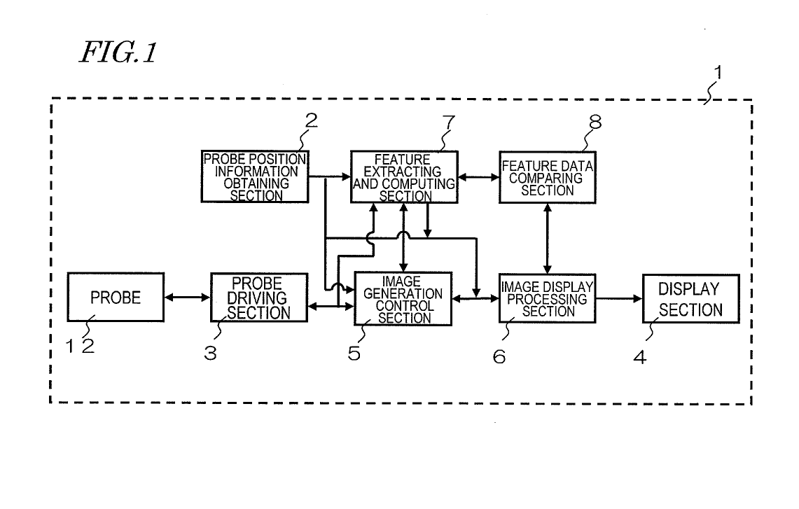

FIG. 1 is a block diagram generally illustrating an embodiment of an ultrasonic diagnostic apparatus according to the present invention.

This ultrasonic diagnostic apparatus 1 includes a probe 12, a probe position information obtaining section 2, a probe driving section 3, an image generation control section 5, a feature extracting and computing section 7, a feature data comparing section 8, an image display processing section 6 and a display section 4. Although the ultrasonic diagnostic apparatus 1 of this embodiment includes the probe 12, the ultrasonic diagnostic apparatus 1 does not have to include the probe 1, but just needs to be connectable to the probe 12.

The probe 12 includes an ultrasonic vibrator, transmits an ultrasonic wave to a subject and receives an echo signal as the reflected ultrasonic wave through the ultrasonic vibrator, and transforms the echo signal into an electrical signal.

The probe position information obtaining section 2 obtains and outputs information about the position of the probe 12.

The probe driving section 3 supplies a drive signal that instructs the probe 12 to transmit an ultrasonic wave as a part of ultrasonic wave transmission processing. Also, as a part of ultrasonic wave reception processing, the probe driving section 3 carries out general reception processing that should be done to compose a tomographic image, including amplifying and detecting the electrical signal supplied from the probe 12, thereby generating a received signal.

The image generation control section 5 performs a coordinate transformation and other kinds of processing on the received signal supplied from the probe driving section 3, thereby generating tomographic image information. In addition, the image generation control section 5 associates the received signal with the positional information that has been obtained by the probe position information obtaining section 2. In this description, the “tomographic image information” refers herein to not only the tomographic image itself that has been composed but also other pieces of information to be used to compose a tomographic image based on the received signal (e.g., brightness information).

The feature extracting and computing section 7 carries out data processing to compose a three-dimensional image of the object of measurement based on the positional information provided by the probe position information obtaining section 2 and the received signal supplied from the probe driving section 3.

The feature data comparing section 8 stores and retains the measurement data, tomographic image and three-dimensional image that were obtained from the object of measurement as a result of a past measurement, and compares them to the output of the feature extracting and computing section 7 that has been obtained as a result of a current measurement.

The image display processing section 6 performs processing to present the tomographic image that has been composed by the image generation control section 5, synthesizes together the data processed by the feature extracting and computing section 7, the tomographic image generated by the image generation control section 5, and the positional information provided by the probe position information obtaining section 2, and controls the display data to present a three-dimensional image of the object of measurement.

The display section 4 displays the output of the image display processing section 6.

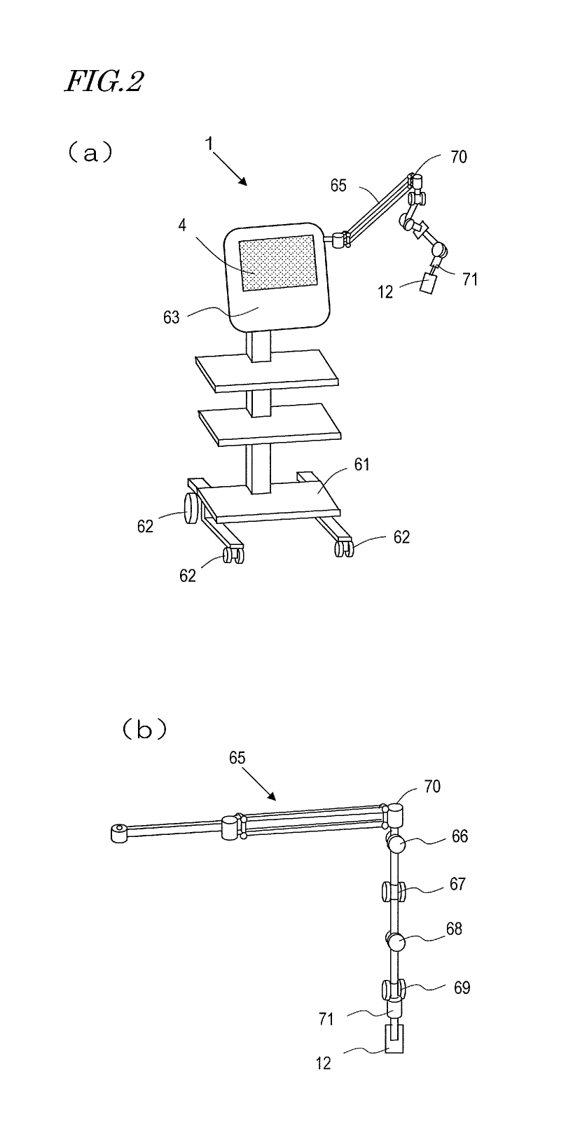

Next, look at FIG. 2, which illustrates an exemplary appearance of the ultrasonic diagnostic apparatus of the present invention.

As shown in FIG. 2(a), the body case 63 of this ultrasonic diagnostic apparatus 1 may be mounted on a caster stage 61 with four tires 62 at its feet, thus making the ultrasonic diagnostic apparatus 1 movable in any direction.

Also, a multi-articulated arm 65 is provided at the rear of the body case 63 of the ultrasonic diagnostic apparatus 1 and the probe 12 is arranged at the end of the arm 65. The probe 12 is electrically connected to the probe driving section 3 in the body case 63.

As shown in FIG. 2(b), the multi-articulated arm 65 includes two rotatable articulations 70 and 71 and a plurality of foldable articulations 66 to 69 which are arranged between the two rotatable articulations 70 and 71. In the configuration illustrated in FIG. 2(b), four articulations are provided. In addition, an angle sensor (not shown) is provided for each of these articulations 66 to 71 to detect its angle of rotation. Furthermore, an acceleration sensor (not shown, either) is also provided for each of the two rotatable articulations 70 and 71 in order to detect the three-dimensional direction acceleration of the articulation.

These angle and acceleration sensors provided for the respective articulations 66 to 71 of the multi-articulated arm 65 together form the probe position information obtaining section 2. Thus, information about the position of the probe 12 is obtained and output using these sensors.

That is to say, the probe 12 can be not only moved up, down, to the right, and to the left and but also freely rotated 360 degrees to any other direction by the multi-articulated arm 65 with those articulations 66 to 71. And information about the position of the probe 12 can be obtained by the angle sensors provided for those articulations of the multi-articulated arm 65. Also, if the multi-articulated arm 65 gets shaken or tilted, the shift caused by the shake or tilt can be detected by the acceleration sensors and the positional information obtained by the angle sensors is corrected based on the magnitude of the shake or tilt.

In this embodiment, the multi-articulated arm 65 including the angle and acceleration sensors and supporting the probe 12 is supposed to be used an exemplary probe position information obtaining section 2. However, this is just an exemplary configuration for obtaining information about the position of the probe 12 and the present invention is in no way limited to this specific configuration. That is to say, as long as information about the position of the probe 12 can be obtained, any other configuration may be used. For example, a magnetic sensor, a gyrosensor, an optical sensor or their combination may also be used. Also, the configuration of the multi-articulated arm 65 is just an embodiment of the present invention to obtain information about the position of the probe 12. Thus, the number of articulations or sensors provided does not have to be the one adopted in the configuration shown in FIG. 2(b).

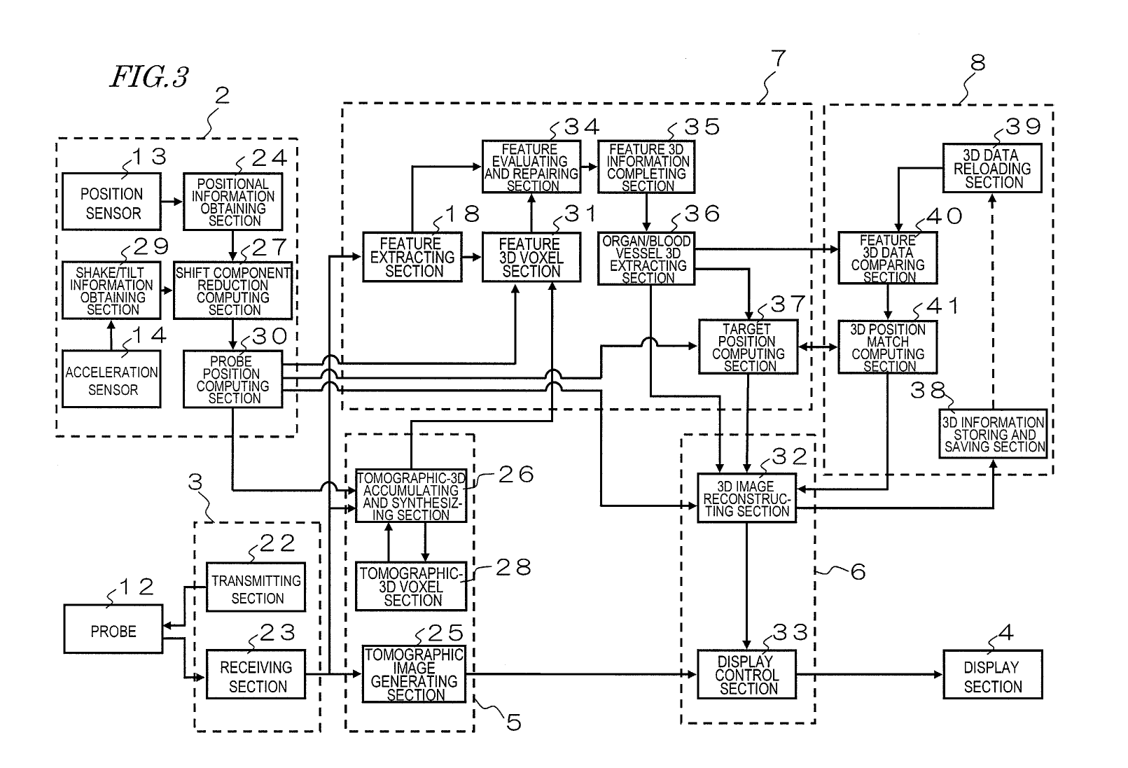

Next, an ultrasonic diagnostic apparatus according to this embodiment will be described in further detail with reference to FIG. 3, which is a detailed block diagram of the ultrasonic diagnostic apparatus 1 shown in FIG. 1. The probe position information obtaining section 2, probe driving section 3, image generation control section 5, feature extracting and computing section 7, feature data comparing section 8, and image display processing section 6 to be described in detail below may be implemented as either pieces of hardware or a combination of an information processor such as a CPU and a software program stored in a storage section such as a memory. In the latter case, the information processor retrieves a software program, defining the procedure of the image getting method to be described below, from the memory and carries out the procedure of the image getting method, thereby controlling the respective components of this ultrasonic diagnostic apparatus. Optionally, some of those components to be implemented as the combination of the information processor and the software program stored in the memory may be implemented as a dedicated integrated circuit as well. Alternatively, some of those components may also be implemented as a general purpose information processor such as a microcomputer. That is to say, this embodiment may be carried out as a system including the ultrasonic diagnostic apparatus and the microcomputer.

As shown in FIG. 3, the probe driving section 3 includes a transmitting section 22 and a receiving section 23. The transmitting section 22 performs the transmission processing described above, and the receiving section 23 performs the reception processing described above.

The probe position information obtaining section 2 includes position sensors 13, a positional information obtaining section 24, acceleration sensors 14, a shake/tilt information obtaining section 29, a shift component reduction computing section 27 and a probe position information computing section 30.

The position sensors 13 are the plurality of angle sensors provided for the multi-articulated arm 65 described above.

The positional information obtaining section 24 obtains information about the position of the probe 12 based on the output of the position sensor 13.

The acceleration sensors 14 are provided for the two rotatable articulations 70 and 71 described above to detect the acceleration of the probe 12 moving.

The shake/tilt information obtaining section 29 obtains information about the shake and tilt of the probe 12 based on the output of the acceleration sensor 14.

The shift component reduction computing section 27 calculates the shift component of the probe 12 by reference to the information about the position of the probe 12 that has been obtained by the positional information obtaining section 24 and the information about the shake and tilt of the probe that has been obtained by the shake/tilt information obtaining section 29.

The probe position computing section 30 calculates information about the exact position of the probe 12 based on the result of the calculation that has been made by the shift component reduction computing section 27.

That is to say, the probe position information obtaining section 2 is configured to get a shift component, which will be noise in obtaining accurate information about the position of the probe 12, calculated by the shift component reduction computing section 27 by reference to not only the information about the shake and tilt of the probe 12 that has been obtained by the shake/tilt information obtaining section 29 based on the output of the acceleration sensor 14 but also the positional information obtained by the positional information obtaining section 24, and to get the accurate position of the probe 12 calculated by the probe position computing section 30 based on the result of the computation. The information about the position of the probe 12 that has been calculated by the probe position computing section 30 is output to a tomographic-3D accumulating and synthesizing section 26, a feature 3D voxel section 31 and a 3D image reconstructing section 32 to be described later. The image generation control section 5 includes a tomographic image generating section 25, the tomographic-3D accumulating and synthesizing section 26, and a tomographic-3D voxel section 28.

The tomographic image generating section 25 generates information about a so-called “general tomographic image” based on the received signal that has been subjected to the reception processing by the receiving section 23.

The tomographic-3D accumulating and synthesizing section 26 associates the received signals, which have been obtained by transmitting and receiving ultrasonic waves to/from multiple points in the subject, with the information provided by the probe position computing section 30. For example, by transmitting and receiving ultrasonic waves while bringing the probe 12 into contact with the surface of the subject and moving the probe 12 in one direction, received signals are obtained from multiple points. In that case, those received signals obtained from the multiple points and multiple pieces of information about the positions of the probe provided by the probe position computing section 30 are sequentially associated with each other one after another.

In this embodiment, the tomographic-3D accumulating and synthesizing section 26 is supposed to associate the received signals with the positions of the probe 12. However, the tomographic-3D accumulating and synthesizing section 26 may also associate the tomographic image information with the probe position information. In that case, the tomographic-3D accumulating and synthesizing section 26 receives the output of the tomographic image generating section 25, instead of the output of the receiving section 23 (not shown in FIG. 3).

The tomographic-3D voxel section 28 gets a plurality of received signals from the tomographic-3D accumulating and synthesizing section 26 and sends back some of those received signals to the tomographic-3D accumulating and synthesizing section 26 as needed.

The feature extracting and computing section 7 includes a feature extracting section 18, the feature 3D voxel section 31, a feature evaluating and repairing section 34, a feature 3D information completing section 35, an organ/blood vessel 3D extracting section 36 and a target position computing section 37.

The feature extracting section 18 extracts data featuring a predetermined object of measurement based on the received signal gotten from the receiving section 23. In this description, the “data featuring a predetermined object of measurement” refers herein to information about the boundary between an organ and the blood vessel or information about a region where there is some structural change to be obtained by analyzing the received signal.

The feature 3D voxel section 31 accumulates the data featuring a plurality of predetermined objects of measurement that have been extracted by the feature extracting section 18, the received signals that have been accumulated by the tomographic-3D accumulating and synthesizing section 26, and information about the position of the probe 12 provided by the probe position computing section 30. Also, in accumulating these pieces of information, the feature 3D voxel section 31 associates these three kinds of information with each other so that these pieces of information are provided for the received signals gotten from multiple points in the subject.

The feature evaluating and repairing section 34 evaluates the extraction status of a predetermined object of measurement based on the data that has been extracted by the feature extracting section 18 and the data that has been accumulated by the feature 3D voxel section 31. Specifically, the feature evaluating and repairing section 34 removes noise components that should be unnatural for a human body structure from the data that has been accumulated in the feature 3D voxel section 31 or repairs the data featuring the predetermined objects of measurement that have been accumulated in the feature 3D voxel section 31 into continuous information.

The feature 3D information completing section 35 completes the information to compose the three-dimensional image of a predetermined object of measurement based on the output of the feature evaluating and repairing section 34.

The organ/blood vessel 3D extracting section 36 extracts information to compose a three-dimensional image of a predetermined region (such as a blood vessel or a specified organ) that has been selected separately in accordance with an instruction from the information to compose a three-dimensional image of a predetermined object of measurement that has been completed by the feature 3D information completing section 35.

The target position computing section 37 updates, from time to time, the relative position of the probe 12 with respect to the predetermined region that has been separately specified by the organ/blood vessel 3D extracting section 36 on the three-dimensional image of that predetermined region by reference to information to compose a three-dimensional image of that predetermined region and information about the position of the probe 12 that has been obtained from the probe position computing section 30. Then, the target position computing section 37 gets the position of the predetermined region that has been subjected to the measurement in the object of measurement stored in a 3D information storing and saving section 38 via the 3D image reconstructing section 32. Also, as will be described in detail later, if the position of the predetermined region that was subjected to measurement in the past (which will be referred to herein as a “target position”) matches the position of the probe 2 that is currently making a measurement, the target position computing section 37 outputs information indicating their match to the 3D image reconstructing section 32 and gives an instruction on how to incorporate the position into the three-dimensional image.

The feature data comparing section 8 includes the 3D information storing and saving section 38, a 3D data reloading section 39, a feature 3D data comparing section 40, and a 3D position match computing section 41.

The 3D information storing and saving section 38 stores the information to compose the three-dimensional image and target position that have been gotten from the 3D image reconstructing section 32.

The 3D data reloading section 39 retrieves the data that is saved in the 3D information storing and saving section 38.

The feature 3D data comparing section 40 compares information to compose a past three-dimensional image that has been retrieved by the 3D data reloading section 39 to the three-dimensional image of the object of measurement that has been extracted by the organ/blood vessel 3D extracting section 36 during the current measurement, and calculates their positional shift component. For example, the feature 3D data comparing section 40 compares information about the structure of the object of measurement covered in the past three-dimensional image to information about the structure of the object of measurement currently under inspection, thereby calculating their positional shift component.

The 3D position match computing section 41 makes computations based on the positional shift component provided by the feature 3D data comparing section 40 to determine whether or not the three-dimensional image that was gotten in the past matches the three-dimensional image being gotten currently. If the answer is YES, the 3D position match computing section 41 outputs that information to the 3D image reconstructing section 32 to be described below to incorporate the target position into the three-dimensional image being gotten currently.

The image display processing section 6 includes the 3D image reconstructing section 32 and a display control section 33.

The 3D image reconstructing section 32 rearranges the tomographic image supplied from the image generation control section 5, the three-dimensional image obtained by the feature extracting and computing section 7, and the positional information that is the output of the probe position information obtaining section 2 so that these images and information are synthesized together and that their synthetic image is displayed on the display section 4. In addition, by reference to the information provided by the 3D position match computing section 41, the 3D image reconstructing section 32 gets the target position that was subjected to measurement in the past displayed on the three-dimensional image being gotten currently. Furthermore, on receiving information indicating that the position of the probe 2 currently used to make a measurement has matched the target position from the target position computing section 37, the 3D image reconstructing section 32 outputs display information indicating that to the display control section 33. Optionally, those images and information may also be rearranged with a result of the comparison that has been made by the feature data comparing section 8 with the past three-dimensional image also taken into account.

The display control section 33 performs a control operation so that the tomographic image generated by the tomographic image generating section 25 and the output of the 3D image reconstructing section 32 can be displayed on the display section 4.

Next, it will be described with reference to FIGS. 4 through 10 how the ultrasonic diagnostic apparatus of this embodiment shown in FIGS. 1 to 3 may operate and how the operator may use it. In the example to be described below, the IMT of the carotid artery is supposed to be measured. Also, in the following example, the procedure of measuring the IMT of the same person under test twice at an interval will be described. In that case, if a measurement is currently being made for the second time, the measurement that was carried out for the first time is a past measurement. Also, if the IMT of the same person under test is measured n times (where n is an integer that is equal to or greater than three) at some intervals, the IMT measurement carried out for the second time in the example to be described below may be an IMT measurement carried out for the n^{th} time.

First of all, it will be described with reference to the flowchart shown in FIG. 4 how the ultrasonic diagnostic apparatus may operate and how the operator may use it in a situation where the IMT of the carotid artery is measured for the first time (i.e., at the time of the first consultation). In Step #1 (S11), the probe 12 is brought into contact with the surface of the subject's nape and his or her carotid artery is searched for based on the tomographic image obtained.

Specifically, with the transmitting section 22 and receiving section 23 of the probe driving section 3 driven, the end portion of the probe 12 is brought into contact with the surface of the nape 16 as shown in FIG. 5(a). In this case, the probe 12 is arranged so that an ultrasonic beam transmitted from the probe 12 strikes the carotid artery perpendicularly to the direction in which the blood vessel extends (which will be referred to herein as the “long-axis direction”) and that a tomographic image representing a substantially circular cross section of the carotid artery (which will be referred to herein as a “short-axis cross section”) can be obtained. That is to say, the operator searches for the carotid artery by moving the probe 12 while keeping the probe 12 facing the same direction so that a short-axis cross section of the carotid artery will be included in the tomographic image. When the probe 12 is arranged at an appropriate position, a substantially circular short-axis cross section 17 of the carotid artery will be included in the tomographic image that has been generated by the tomographic image generating section 25 and the display control section 30 and that is now displayed on the display section 4, as in the tomographic image 17 shown in FIG. 5(b).

Next, in Step #2 (S12), received signals are gotten from multiple points on the carotid artery in the long-axis direction.

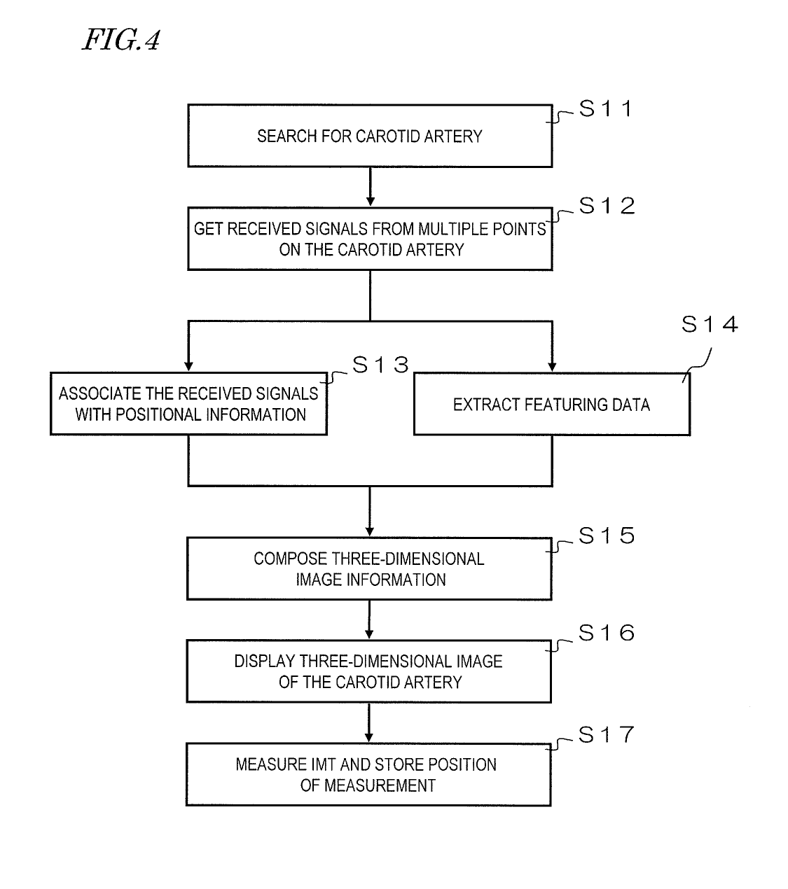

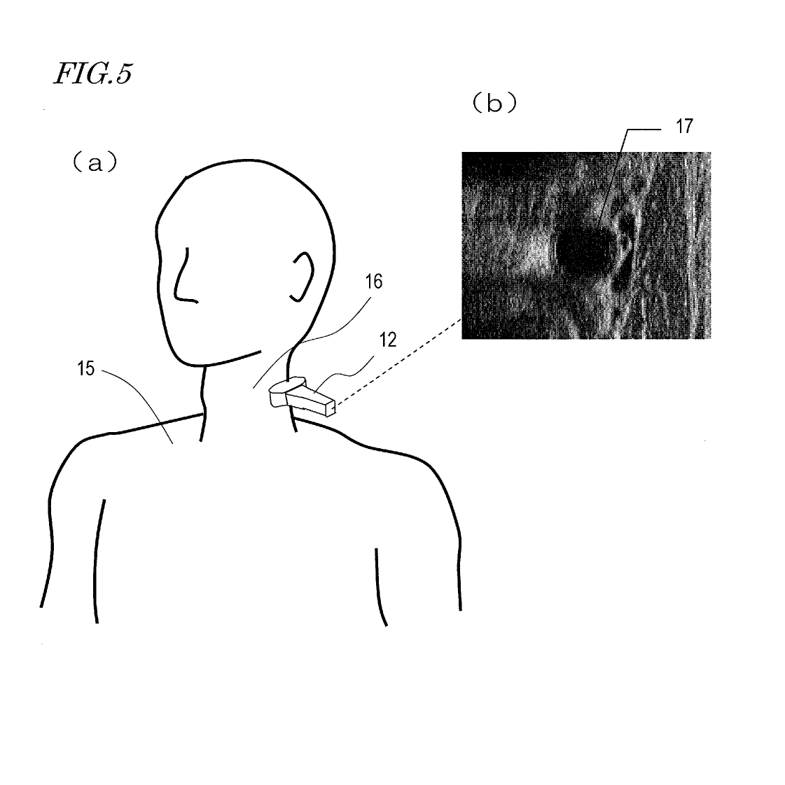

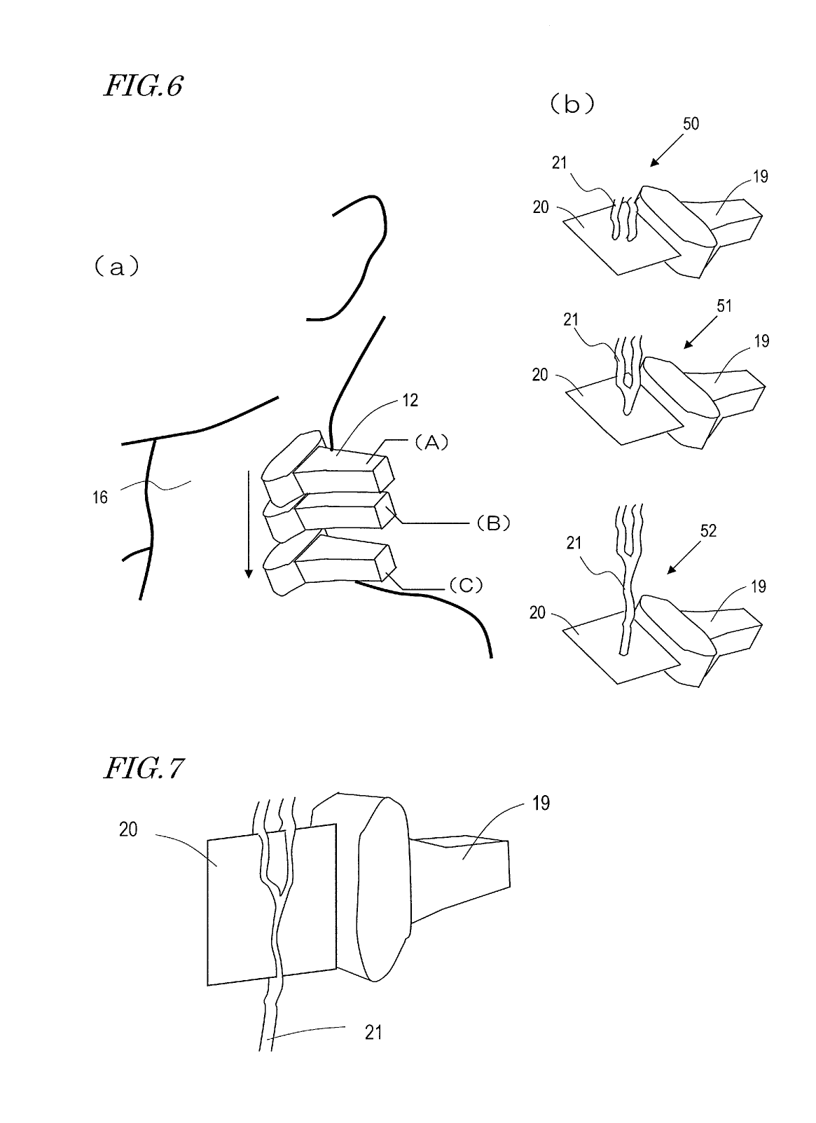

With the probe 12 brought into contact with the surface of the nape at an appropriate position in Step #1 (S11), the probe 12 is moved along the long axis of the carotid artery as indicated by the arrow in FIG. 6(a), for example. And as the probe 12 is moved, ultrasonic waves are transmitted to, and echo signals are received as reflected ultrasonic waves from, multiple points on the carotid artery in the long-axis direction (e.g., the points (A), (B) and (C) shown in FIG. 6(a)). In this description, this operation will be referred to herein as “hand scanning”. As a result, echo signals representing the short-axis cross section of the carotid artery can be received from multiple points on the carotid artery in the long-axis direction.

In this embodiment, in receiving echo signals representing the short-axis cross section of the carotid artery from multiple points on the carotid artery in the long-axis direction, hand scanning is supposed to be performed. However, this is just an example of the present invention. Alternatively, a so-called “3D probe” or “4D probe” may also be used as the probe. In that case, echo signals representing the short-axis cross section of the carotid artery are received electronically, not by hand scanning, from multiple points on the carotid artery in the long-axis direction.

Steps #3 (S13) through #6 (S16) to be described below are performed substantially simultaneously with Step #2 (S12) described above to get a three-dimensional image of the carotid artery displayed on the display section 4. It should be noted that these Steps #3 (S13) through #6 (S16) are carried out sequentially while making hand scanning.

In Step #3 (S13), multiple received signals obtained from respective points on the carotid artery in the long-axis direction by hand scanning get associated with information about the position of the probe 12 that has gotten those received signals.

First of all, those received signals obtained from the respective points by hand scanning are sequentially output from the receiving section 23 to the tomographic-3D accumulating and synthesizing section 26. In the meantime, information about the position of the probe 12 that has gotten these received signals is obtained by the probe position information obtaining section 2. Then, the tomographic-3D accumulating and synthesizing section 26 associates those received signals and the positional information with each other. And the information thus associated is stored in the tomographic-3D voxel section 28.

In Step #4 (S14), data featuring the predetermined object of measurement is extracted from the received signals that have been obtained from the respective points by hand scanning.

First of all, as in Step #3 (S13), those received signals obtained by hand scanning from the respective points on the carotid artery in the long-axis direction are sequentially output from the receiving section 23 to the feature extracting section 18. Based on those received signals, the feature extracting section 18 gets boundary information from a blood vessel including the carotid artery or from any other organ. Such boundary information may be obtained by a vascular boundary detecting method which is generally used in measuring the IMT. Specifically, in that case, data with predetermined amplitude or more is extracted by reference to information about the amplitude of the received signal and used as the boundary information. This boundary information is obtained every time a received signal is output from the receiving section 23 to the feature extracting section 18.

It should be noted that this boundary information is original data to compose a three-dimensional image. By performing various kinds of data processing in Step #5 (S15) (to be described later) with multiple pieces of boundary information gotten from respective points on the carotid artery combined together, a three-dimensional image can be composed.

In Step #5 (S15), information to compose the three-dimensional image is completed.

In this example, the information is repaired into continuous information by the feature evaluating and repairing section 34 based on the data obtained by the feature extracting section 18 and accumulated in the feature 3D voxel section 31 and the information provided by the tomographic-3D accumulating and synthesizing section 26. After that, the information to compose a three-dimensional image of the predetermined object of measurement, including the carotid artery, is completed by the feature 3D information completing section 35.

In Step #6 (S16), a three-dimensional image representing the carotid artery as the object of measurement is selectively displayed based on the information to compose a three-dimensional image that has been completed in Step #5 (S15). The information to compose a three-dimensional image that has been obtained in Step #5 (S15) includes not only information about the carotid artery but also information about other blood vessels and organs as well. That is why in Step #6 (S16), only information about the carotid artery is extracted from those pieces of information to compose the three-dimensional image that has been obtained.

The carotid artery is thicker than an ordinary blood vessel and has a structure consisting of a common carotid artery, an inner carotid artery, and an outer carotid artery. And the carotid artery has a characteristic Y-structure in which the common carotid artery extends toward the head of the subject and branches into the inner and outer carotid arteries (i.e., a so-called “common carotid artery branching portion”).

That is why information about such a characteristic structure and the thickness of a standard carotid artery is stored in advance in the organ/blood vessel 3D extracting section 36 and a three-dimensional image representing the carotid artery is selected according to its characteristic structure from the three-dimensional image that has been completed in Step #5 (S15). Information about the carotid artery three-dimensional image thus selected is output to the 3D image reconstructing section 32, which composes a three-dimensional image representing only the carotid artery based on the information gotten. Then, the three-dimensional image representing only the carotid artery is displayed on the display section 4 via the display control section 33.

In the case of the carotid artery, almost as soon as hand scanning is started, the object can usually be quickly recognized as the carotid artery by its structure and thickness (i.e., blood vessel diameter) and such a three-dimensional image representing only the carotid artery can be displayed. However, if there were two or more candidate objects, any one of which could be the carotid artery, and if it is impossible to decide which one of them is actually the object to present its three-dimensional image, then images of all of those candidate objects may be displayed on the display section 4, and compared to the information about the structure of the carotid artery from time to time by reference to the information gotten by hand scanning, thereby deleting objects that cannot be the carotid artery.

By performing these processing steps #3 (S13) through #6 (S16), a tomographic image obtained by hand scanning and a three-dimensional image of the carotid artery such as the one shown in FIG. 6(b) are displayed almost synchronously with each other on the display section 4.

In presenting a three-dimensional image such as the one shown in FIG. 6(b), the image display processing section 6 of the ultrasonic diagnostic apparatus of this embodiment superimposes information about the position of the probe obtained from the probe position information obtaining section 2 on the three-dimensional image and gets the three-dimensional image with the positional information displayed on the display section 4 so as to allow even an unskilled operator to understand easily the status of the three-dimensional image gotten. More specifically, a schematic representation 19 of the probe 12 and a partially quadrangle region 20 showing a major tomographic image getting area at the scanning plane position at the end of the schematic representation 19 are synthesized together, and the synthetic image thus obtained is displayed so as to be superimposed on the three-dimensional image (the combined image which will also be referred to herein as a “synthetic image”). As will be described later, this synthetic image will be a guide when hand scanning is carried out.

Subsequently, as this probe 12 is moved downward along the nape 16 as indicated by the arrow in FIG. 6(a), tomographic images (not shown) are obtained from the points (A), (B) and (C) shown in FIG. 6(a).

By performing these processing steps #3 (S13) through #6 (S16), the three-dimensional images 50, 51 and 52 of the carotid artery 21 are synthesized and accumulated and displayed in the order of (A), (B) and (C) on the partially quadrangle region 20 of the synthetic image on the display monitor 4 of the ultrasonic diagnostic apparatus 1 almost synchronously with hand scanning.

When the three-dimensional image of the carotid artery gets composed and displayed by performing these processing steps, the process advances to Step #7 (S17) of measuring the IMT. The IMT may be measured by applying a general known IMT measuring technique to the ultrasonic diagnostic apparatus of this embodiment. That is why no configuration for measuring the IMT is shown in FIG. 1 or 3. However, unlike when the IMT is measured by a conventional technique, information about the position where the IMT has been measured is also obtained by the ultrasonic diagnostic apparatus of this embodiment.

Before this Step #7 (S17) is described, it will be described briefly how to measure the IMT using an ultrasonic diagnostic apparatus.

In general, the IMT is measured by detecting the boundary between the vascular lumen and intima of the carotid artery (which will be referred to herein as a “lumen-intima boundary”) and the boundary between the media and adventitia thereof (which will be referred to herein as a “media-adventitia boundary”) and measuring the distance between the lumen-intima boundary and the media-adventitia boundary based on a received signal or tomographic image representing a cross section of the carotid artery as viewed in the long-axis direction (which will be referred to herein as a “long-axis cross section”). Then, a predetermined measuring range of the vascular wall is set to be a region of interest and either the maximum thickness (max IMT) or average thickness (mean IMT) within the region of interest is calculated as the IMT value. In this case, they say that the region of interest is preferably defined within 1 cm away from the far end (closer to the head) of the common carotid artery (see Journal of the American Society of Ethocardiography, February 2008 (pp. 93 to 111)).

To measure the IMT, first of all, a short-axis image is displayed and the carotid artery is located by hand scanning described above. Next, when decision is made, based on the structure of the carotid artery branching portion, that the probe 12 is arranged by the operator roughly within the range where the region of interest should be set, the orientation of the probe 12 shown in FIG. 5(a) is changed at that position so that the probe 12 is in upright position (i.e., parallel to the long-axis direction of the carotid artery) as shown in FIG. 7, and a long-axis image is displayed. Thereafter, with the probe 12 held at that upright position, either a received signal or tomographic image representing a long-axis cross section of the carotid artery is obtained to measure the IMT.

Then, in Step #7 (S17), the positions where the IMT has been measured are obtained by the target position computing section 37, and are stored, along with the three-dimensional image obtained in Steps #3 (S13) to #6 (S16), in the 3D information storing and saving section 38. That is to say, the positional information of the probe 12 in the upright position that has been obtained by the probe position information obtaining section 2 and the positional information of the carotid artery, of which the IMT has been measured, as in the partially quadrangle region 20 with respect to the carotid artery shown in FIG. 7 are stored along with the three-dimensional image of the carotid artery.

In this case, if the ultrasonic diagnostic apparatus is configured to store the result of the IMT measurement along with those pieces of information, then the ultrasonic diagnostic apparatus will come in even handier, because the result of a past measurement (e.g., measurement for the first time around) can be seen easily when the IMT is measured for the second time and on (i.e., at the time of re-inspection).

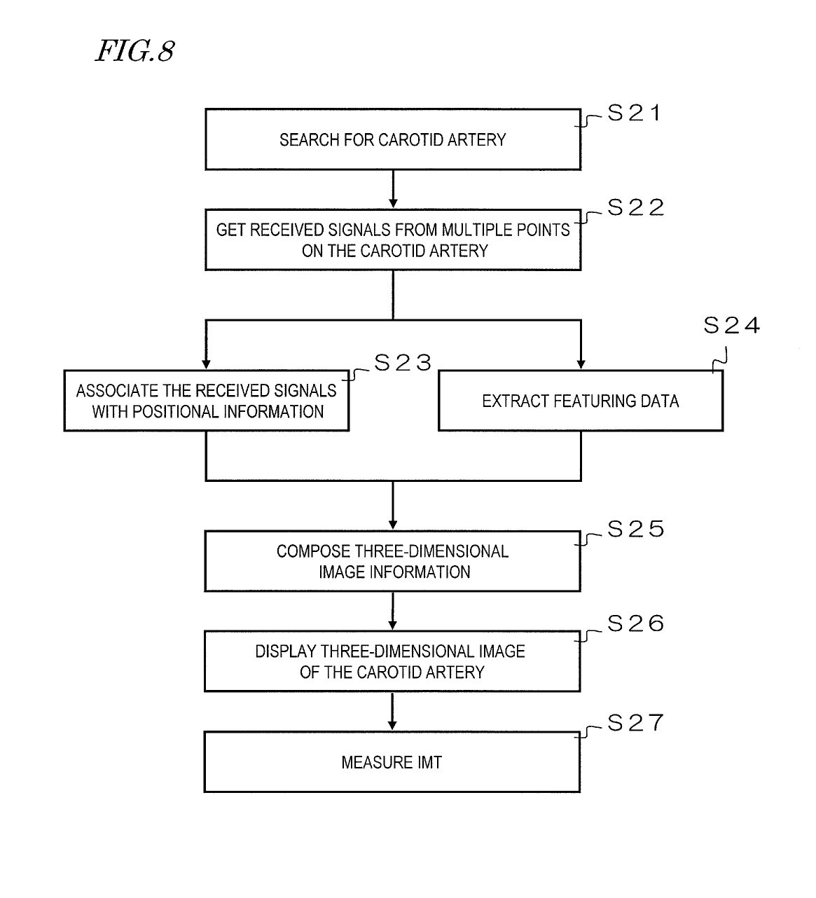

Next, it will be described with reference to the flowchart shown in FIG. 8 how the ultrasonic diagnostic apparatus may operate and how the operator may use the apparatus when measuring the IMT of the carotid artery for the second time (and for the third time and on).

Steps #1 (S21) through #5 (S25) shown in FIG. 8 respectively correspond to Steps #1 (S11) through #5 (S15) shown in FIG. 4 and the same operations are performed in those two series of processing steps. Thus, description thereof will be omitted herein and only Step #6 (S26) and the rest of the procedure will be described herein.

In Step #6 (S26), a three-dimensional image of the carotid artery as the object of measurement is selectively displayed by reference to the information to compose the three-dimensional image that has been completed in the previous processing step #5 (S25). This processing step #6 (S26) is the same as the processing step #5 (S15) shown in FIG. 4. That is why description of such an operation of selectively displaying the three-dimensional image of the carotid artery when the measurement is made for the second time will be omitted herein.

The difference is that not only the three-dimensional image is selectively displayed but also the position where the IMT has been measured for the first time is incorporated into the three-dimensional image that is selectively displayed as a result of the inspection for the second time around.

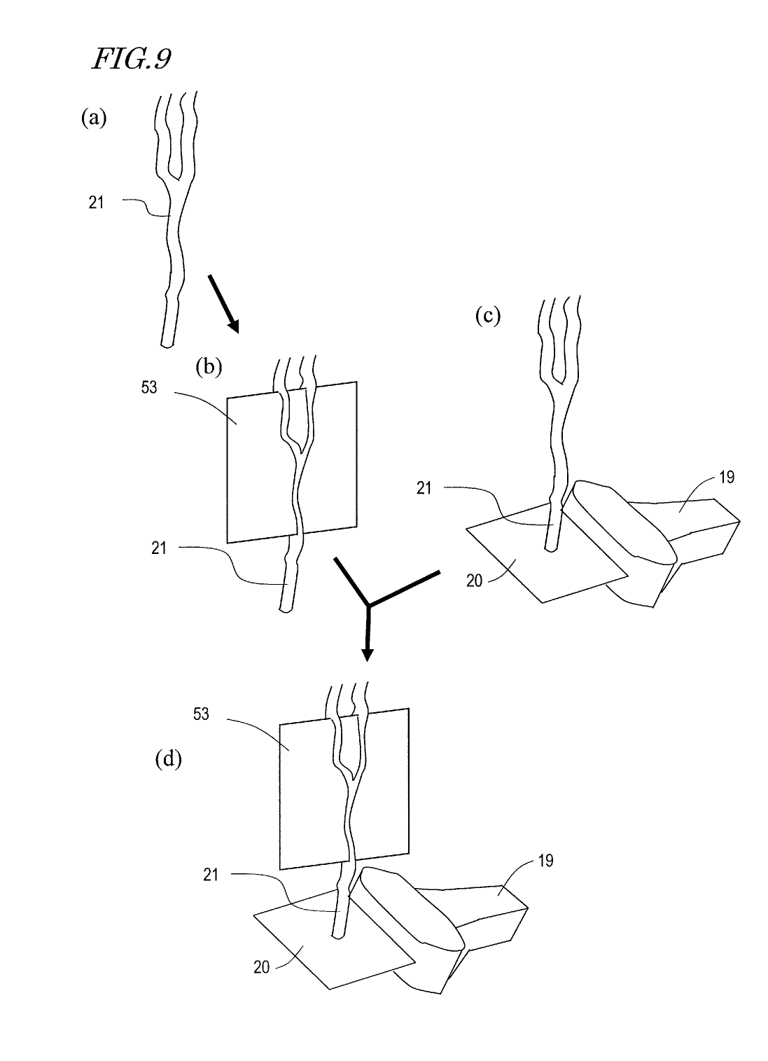

Specifically, first of all, the information to compose the three-dimensional image of the carotid artery and the information about the IMT measuring point on the carotid artery that have been stored in the 3D information storing and saving section 38 as a result of the inspection for the first time are loaded by the 3D reloading section 39 and output to the feature 3D data comparing section 40 (see FIGS. 9(a) and 9(b)).

When the inspection is carried out for the second time, the information to compose the three-dimensional image of the carotid artery that has been extracted by the organ/blood vessel 3D extracting section 36 (see FIG. 9(c)) and the information to compose the three-dimensional image that has been subjected to the IMT measurement for the first time, which has been output to the feature 3D data comparing section 40 (see FIG. 9(a)), are compared to each other by reference to the characteristic structure of the carotid artery such as the Y-structure that is the branching portion of the carotid artery. And based on the result of this comparison, the 3D position match computing section 41 makes computations to determine whether or not the information to compose the three-dimensional image for the first time and the information to compose the three-dimensional image for the second time match each other.

If the decision is made, based on the result of the computations carried out by the 3D position match computing section 41, that the information to compose the three-dimensional image for the first time and the information to compose the three-dimensional image for the second time match each other, then the 3D position match computing section 41 instructs the target position computing section 37 to notify the 3D image reconstructing section 32 of the position where the IMT of the carotid artery has been measured for the first time (see FIG. 9(b)) in order to incorporate the position where the IMT of the carotid artery has been measured for the first time (i.e., the partially quadrangle region 53 shown in FIG. 9(b)) into the information to compose the three-dimensional image that has been gotten as a result of the measurement for the second time. For example, by indicating the position where the IMT of the carotid artery has been measured for the first time as the partially quadrangle region 53 on the three-dimensional image of the carotid artery that has been gotten as a result of the measurement for the second time (see FIG. 9(c)), the position is incorporated into the three-dimensional image (see FIG. 9(d)). And the 3D position match computing section 41 makes computations to determine whether or not the three-dimensional image that was gotten in the past and the three-dimensional image being gotten currently match each other. If the answer is YES, the 3D position match computing section 41 provides that information for the 3D image reconstructing section 32 (to be described later) in order to incorporate the target position into the three-dimensional image being gotten currently.

Next, the IMT is measured in Step #7 (S27).

The processing step of measuring the IMT is carried out in the same way as described above, and description thereof will be omitted herein. The point in this Step #7 (S27) is the operation of moving the probe 12 to the target position.

When the three-dimensional image that has been gotten as a result of the measurement for the second time and the partially quadrangle region 53 that is the target position incorporated into the three-dimensional image are displayed, the operator moves the probe 12 to the vicinity of the target position while looking at the partially quadrangle region 53 displayed on the display section 4. In this case, by reference to the information provided by the probe position information obtaining section 2, the schematic representation of the probe 12 and the partially quadrangle region 20 indicating a main region where the tomographic image is obtained (synthetic image) with respect to the three-dimensional image being displayed are displayed on the display section 4 while being updated from time to time.

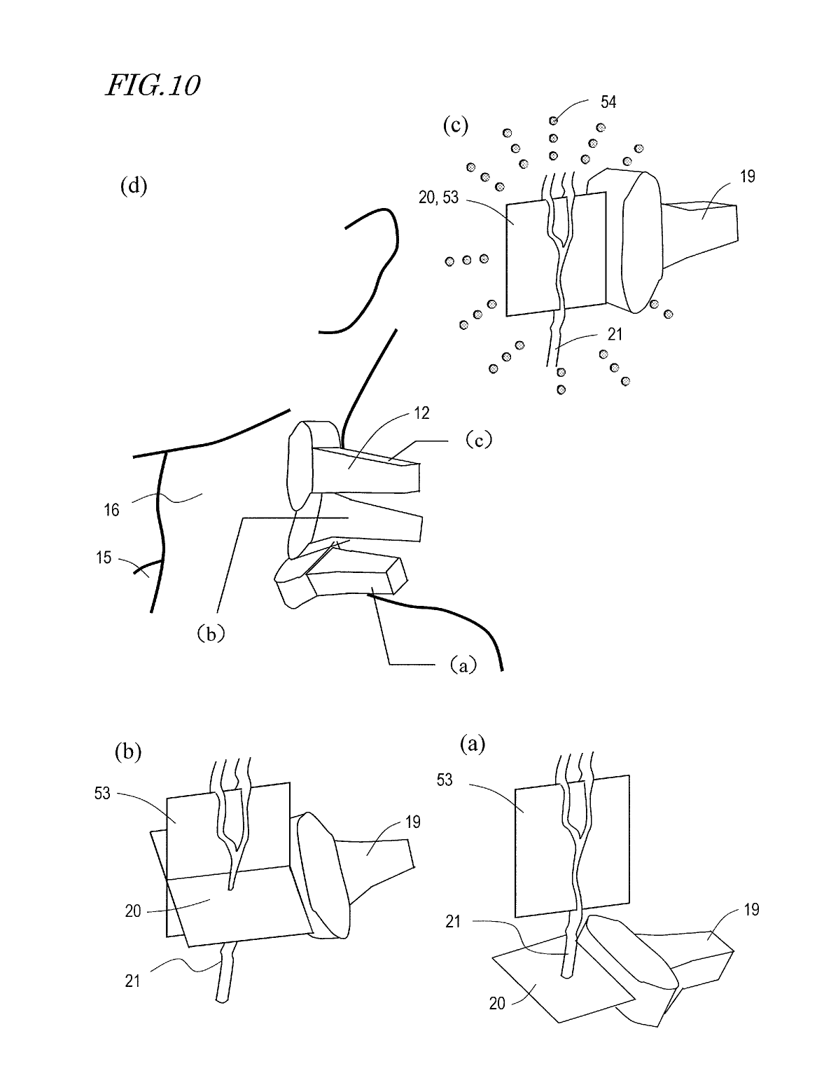

When the probe 12 is moved to the vicinity of the target position, the operator changes the orientation of the probe 12 so that the probe 12 is in an upright position (i.e., parallel to the long axis of the carotid artery) as shown in FIGS. 10(a) and 10(b). In this case, by reference to the information provided by the probe position information obtaining section 2, the synthetic image of the schematic representation 19 of the probe 12 and the partially quadrangle region 20 is displayed on the display section 4 while having its orientation updated from time to time.

And when the partially quadrangle region 20 indicating the position where the measurement has been carried out for the second time matches the partially quadrangle region 53 indicating the position where the measurement was carried out for the first time as shown in FIG. 10(c), the target position computing section 37 instructs the 3D image reconstructing section 32 to indicate their match. In accordance with the instruction, the 3D image reconstructing section 32 gives a sign of match 54 such as the one shown in FIG. 10(c) on the display section 4, thereby notifying the operator of their match. After that, the IMT is measured as described above.

As can be seen from the foregoing description, the ultrasonic diagnostic apparatus of this embodiment can not only generate a three-dimensional image as an ultrasonic image but also compose a three-dimensional image by extracting only an image of any intended blood vessel or organ. Also, this three-dimensional image and the tomographic image gotten share the same positional information. That is why the position where a measurement was carried out in the past is incorporated into, and displayed on, the three-dimensional image gotten by current measurement, thus realizing an ultrasonic diagnostic apparatus with very good operability.

### INDUSTRIAL APPLICABILITY

With an ultrasonic diagnostic apparatus and method of getting an image using an ultrasonic wave according to the present disclosure, the position of the probe can be adjusted to a predetermined position by performing a series of simple operations, thus realizing good operability. That is to say, only an image of an intended object of measurement such as a blood vessel or a viscera can be extracted as a three-dimensional image. On top of that, this three-dimensional image and the tomographic image share the same positional information.

That is why if some region of interest is specified on the three-dimensional image of the object of measurement, a tomographic image of that region can be checked out easily. Consequently, an ultrasonic diagnostic apparatus and method of getting an image using an ultrasonic wave according to the present disclosure can be used effectively not just to measure the IMT but also for various other medical inspection and diagnosis purposes.

### REFERENCE SIGNS LIST

1 ultrasonic diagnostic apparatus
2 probe position information obtaining section
3 probe driving section
4 display section
5 image generation control section
6 image display processing section
7 feature extracting and computing section
8 feature data comparing section
12 probe
13 position sensor
14 acceleration sensor
15 subject
16 nape
17 short-axis cross section
18 feature extracting section
19 schematic representation of probe
20, 53 partially quadrangle region
21 carotid artery
22 transmitting section
23 receiving section
24 positional information obtaining section
25 tomographic image generating section
26 tomographic-3D accumulating and synthesizing section
27 shift component reduction computing section
28 tomographic-3D voxel section
29 shake/tilt information obtaining section
30 probe position computing section
31 feature 3D voxel section
32 3D image reconstructing section
33 display control section
34 feature evaluating and repairing section
35 feature 3D information completing section
36 organ/blood vessel 3D extracting section
37 target position computing section
38 3D information storing and saving section
39 3D data reloading section
40 feature 3D data comparing section
41 3D position match computing section
50, 51, 52 three-dimensional image of carotid artery
54 sign of match
61 caster stage
62 tire
63 body case
65 multi-articulated arm
66 to 71 articulation

1. An ultrasonic diagnostic apparatus which is connectable to a probe that transmits and receives an ultrasonic wave and that converts its echo signal into an electrical signal and which measures a characteristic of a subject in a predetermined region of its object of measurement, the apparatus comprising:
a probe driving section configured:
to perform transmission processing to transmit the ultrasonic wave and reception processing to generate a received signal based on the electrical signal by driving the probe and
to get a received signal from each point of the subject including the object of measurement;
a probe position information obtaining section configured to obtain positional position information about the probe that has gotten the received signal from each said point;
an image generation control section configured to associate the received signal from each said point with the positional information;
a feature extracting and computing section configured to generate, based on the received signal with which the positional information has been associated, information to compose a three-dimensional image covering the object of measurement;
a feature data comparing section configured:
to store information to compose a three-dimensional image that was gotten in the past and information about the position of the object of measurement, of which the characteristic was measured in the past,
to compare the information to compose the three-dimensional image that was gotten in the past to information to compose a three-dimensional image being gotten currently, and
to get the positional information of the object of measurement, of which the characteristic was measured in the past, incorporated into the information to compose the three-dimensional image being gotten currently;
an image display processing section configured to perform processing to display a three-dimensional image based on the information to compose the three-dimensional image being gotten currently, the positional information of the object of measurement, of which the characteristic was measured in the past and which has been incorporated by the feature data comparing section, and information about the current position of the probe; and
a display section configured to display the output of the image display processing section.

2. An ultrasonic diagnostic apparatus which is connectable to a probe that transmits and receives an ultrasonic wave and that converts its echo signal into an electrical signal and which measures a characteristic of a subject in a predetermined region of its object of measurement, the apparatus comprising:
a probe driving section configured:
to perform transmission processing to transmit the ultrasonic wave and reception processing to generate a received signal based on the electrical signal by driving the probe and
to get a received signal from each point of the subject including the object of measurement;
a probe position information obtaining section configured to obtain positional information about the probe that has gotten the received signal from each said point;
an image generation control section configured:
to generate tomographic image information based on the received signal from each said point and
to associate the tomographic image information with the positional information;
a feature extracting and computing section configured to generate, based on the tomographic image information with which the positional information has been associated, information to compose a three-dimensional image covering the object of measurement;
a feature data comparing section configured:
to store information to compose a three-dimensional image that was gotten in the past and information about the position of the object of measurement, of which the characteristic was measured in the past,
to compare the information to compose the three-dimensional image that was gotten in the past to information to compose a three-dimensional image being gotten currently, and
to get the positional information of the object of measurement, of which the characteristic was measured in the past, incorporated into the information to compose the three-dimensional image being gotten currently;
an image display processing section configured to perform processing to display a three-dimensional image based on the information to compose the three-dimensional image being gotten currently, the positional information of the object of measurement, of which the characteristic was measured in the past and which has been incorporated by the feature data comparing section, and information about the current position of the probe; and
a display section configured to display the output of the image display processing section.

3. The ultrasonic diagnostic apparatus of claim 1, wherein the image display processing section superimposes the information about the current position that has been obtained by the probe position information obtaining section on the three-dimensional image based on the information to compose the three-dimensional image being gotten currently and then displays the three-dimensional image with the current position information on the display section.

4. The ultrasonic diagnostic apparatus of claim 1, comprising a storage section configured to store the information to compose the three-dimensional image that was gotten in the past and the positional information of the object of measurement, of which the characteristic was measured in the past.

5. The ultrasonic diagnostic apparatus of claim 1, wherein if the feature data comparing section has found that the probe's current position with respect to the object of measurement matches the position of the object of measurement, of which the characteristic was measured in the past, the feature extracting and computing section instructs the image display processing section to give a sign of their match on the display section.

6. The ultrasonic diagnostic apparatus of claim 1, wherein the feature extracting and computing section extracts information to compose the three-dimensional image of the object of measurement from pieces of information to compose the three-dimensional image by reference to information about the structure of the object of measurement.

7. The ultrasonic diagnostic apparatus of claim 6, wherein the image display processing section gets the information to compose the three-dimensional image of the object of measurement, which has been extracted by the feature extracting and computing section, displayed on the display section.

8. The ultrasonic diagnostic apparatus of claim 1, wherein the feature data comparing section compares the information to compose the three-dimensional image that was gotten in the past and the information to compose the three-dimensional image being gotten currently by reference to the information about the structure of the object of measurement.

9. The ultrasonic diagnostic apparatus of claim 1, wherein the object of measurement is a carotid artery and the characteristic to measure is a characteristic of a blood vessel.

10. The ultrasonic diagnostic apparatus of claim 9, wherein the characteristic of the blood vessel to measure is IMT.

11. The ultrasonic diagnostic apparatus of claim 9, wherein the feature data comparing section compares the information to compose the three-dimensional image that was gotten in the past to the information to compose the three-dimensional image being gotten currently based on the structure of a common carotid artery branching portion where the common carotid artery of the carotid artery branches into an inner carotid artery and an outer carotid artery.

12. The ultrasonic diagnostic apparatus of claim 9, wherein the feature data comparing section compares the information to compose the three-dimensional image that was gotten in the past to the information to compose the three-dimensional image being gotten currently based on the blood vessel diameter of the carotid artery.

13. An image getting method using an ultrasonic wave which measures a characteristic of a subject in a predetermined region of its object of measurement by using a probe that transmits and receives the ultrasonic wave and that converts its echo signal into an electrical signal, the method comprising the steps of:
(A) performing transmission processing to transmit the ultrasonic wave and reception processing to generate a received signal based on the electrical signal by driving the probe and getting a received signal from each point of the subject including the object of measurement;
(B) obtaining positional information about the probe that has gotten the received signal from each said point;
(C) associating the received signal from each said point, including the object of measurement, with the positional information;
(D) generating, based on the received signal with which the positional information has been associated, information to compose a three-dimensional image covering the object of measurement;
(E) comparing the information to compose the three-dimensional image that was gotten in the past to information to compose a three-dimensional image being gotten currently, and getting the positional information of the object of measurement, of which the characteristic was measured in the past, incorporated into the information to compose the three-dimensional image being gotten currently; and
(F) performing processing to display a three-dimensional image based on the information to compose the three-dimensional image being gotten currently, the positional information of the object of measurement, of which the characteristic was measured in the past, and information about the current position of the probe.

14. An image getting method using an ultrasonic wave which measures a characteristic of a subject in a predetermined region of its object of measurement by using a probe that transmits and receives the ultrasonic wave and that converts its echo signal into an electrical signal, the method comprising the steps of:
(A′) performing transmission processing to transmit the ultrasonic wave and reception processing to generate a received signal based on the electrical signal by driving the probe and getting a received signal from each point of the subject including the object of measurement;
(B′) obtaining positional information about the probe that has gotten the received signal from each said point;
(C′) generating tomographic image information based on the received signal from each said point, including the object of measurement, and associating the tomographic image information with the positional information;
(D′) generating, based on the tomographic image information with which the positional information has been associated, information to compose a three-dimensional image covering the object of measurement;
(E′) comparing the information to compose the three-dimensional image that was gotten in the past to information to compose a three-dimensional image being gotten currently, and getting the positional information of the object of measurement, of which the characteristic was measured in the past, incorporated into the information to compose the three-dimensional image being gotten currently; and
(F′) performing processing to display a three-dimensional image based on the information to compose the three-dimensional image being gotten currently, the positional information of the object of measurement, of which the characteristic was measured in the past, and information about the current position of the probe.
